(19) United States
(12) Patent Application Publication
(10) Pub. No.: US 2023/0414749 A1
(43) Pub. Date: Dec. 28, 2023

(54) USE OF IMMUNE CHECKPOINT INHIBITORS IN COMBINATION WITH ANTI-AGING DRUGS IN PREPARATION OF TUMOR TREATMENT PRODUCTS

(71) Applicant: SUN YAT-SEN MEMORIAL HOSPITAL, SUN YAT-SEN UNIVERSITY, Guangzhou (CN)

(72) Inventors: Song FAN, Guangzhou (CN); Niu LIU, Guangzhou (CN); Jiaying WU, Guangzhou (CN); Tingting CAI, Guangzhou (CN); Qunxing LI, Guangzhou (CN)

(21) Appl. No.: 17/877,020

(22) Filed: Jul. 29, 2022

(30) Foreign Application Priority Data
Jun. 23, 2022 (CN) 202210728020.5

(51) Int. Cl. A61K 39/395; A61K 31/506; A61K 31/352; A61P 35/00

(52) U.S. Cl. CPC A61K 39/3955; A61K 31/506; A61K 31/352; A61P 35/00

(57) ABSTRACT

Use of immune checkpoint inhibitors in combination with anti-aging drugs in tumor treatment and tumor treatment products are disclosed, which provides a new drug and treatment strategy for effective treatment of tumors. The therapeutic effect of the provided treatment of ICIs in combination with the anti-aging drug is superior to that of the current treatment of ICIs treatment alone, and ICIs in combination with chemotherapy, etc. In the action process of ICIs in combination with the anti-aging drug, aging cells can be selectively removed, the aging state of an immune system thus is reversed. Accordingly, the inhibitory effect of tumor on the immune micro-environment is reduced, thus improving the treatment effect on various solid tumors. Based on the use or product provided by the present invention, new strategies and new ideas can be provided for further improving the clinical efficacy and prognosis of the solid tumors.

### CROSS REFERENCE TO RELATED APPLICATIONS

The present application claims priority from Chinese Patent Application No. 202210728020.5 filed on Jun. 23, 2022, all of which are hereby incorporated herein by reference.

### TECHNICAL FIELD

The present invention relates to the technical field of biological medicine and more particularly, relates to a use of immune checkpoint inhibitors in combination with anti-aging drugs in preparation of tumor treatment products.

### BACKGROUND

In the prior art, a treatment method based on immune checkpoint inhibitors (ICIs) includes immune checkpoints inhibitors in combination with chemotherapy, which achieves good curative effects in various solid tumors. However, there are still some patients free from benefit from it, and even incur disease progression. Therefore, the mechanism of the treatment method is required for further study to find new treatment strategies and benefit more patients.

Cell senescence refers to the permanent cell cycle arrest state of normally proliferating cells when subjected to stress responses such as exhaustion of replication and DNA damage. Senescent cells are resistant to apoptosis and secrete a series of pro-inflammatory factors and proteases, known as senescence-associated secretory phenotype (SASP). SASP recruits inflammatory cells to remodel the extracellular matrix, which triggers inappropriate cell death, induces fibrosis and inhibits the stem cell function. Selectively eliminating the senescent cells with anti-aging drugs or destructing SASP with anti-aging drugs are thus potential strategies to widely combat age-related diseases. Present studies on aging of an immune system have shown that aging of the immune system can lead to damage to various tissues throughout the body, and there may be complex actions between immunosenescence and tumor progression. Numerous factors are involved between aging of the immune system and tumor progression, such as the influence of cyclic adenosine monophosphate (cyclic AMP or 3′,5′-cyclic adenosine monophosphate, cAMP), glucose competition, oncogenic responses in the tumor micro-environment. Such factors can induce the senescence of T cells, macrophages, natural killer cells and dendritic cells. These senescent immune cells may be closely related to tumor progression.

### SUMMARY

The present invention thus provides a use of immune checkpoint inhibitors in combination with anti-aging drugs in tumor treatment or preparation of tumor treatment products, and further provides a new drug and treatment strategy for effective treatment of tumors, which can benefit more tumor patients.

In one aspect, a use of immune checkpoint inhibitors in combination with anti-aging drugs in tumor treatment is provided according to the present invention. Specifically, a drug combination method for the treatment of solid tumors is provided, i.e. ICIs in combination with the anti-aging drug. According to one or more embodiments of the present invention, inventors have found that superior anti-cancer effects and safety are achieved in the case where the immune checkpoint inhibitor and the anti-aging drug are administered in combination to an individual having cancer, as compared with the conventional case where the immune checkpoint inhibitor is administered alone or the immune checkpoint inhibitor and cisplatin are administered in combination.

In treatment by applying the immune checkpoint inhibitors (ICIs) in combination with the anti-aging drug, aging cells can be selectively removed, SASP in a tumor micro-environment thus is reduced. Accordingly, the immunosuppressive micro-environment in the tumors is improved and the aging state of an immune system is reversed. Considering the action of the immune checkpoint inhibitors, the inhibitory effect of tumor cells themselves on immune cells is further reduced, thereby improving the effect of tumor treatment. That is, by applying ICIs in combination with the anti-aging drug, the anti-tumor response can be significantly improved and the survival rate of patients can be better improved easily. According to one or more embodiments of the present invention, in a model for treating mouse tumor, by selecting the immune checkpoint inhibitor in combination with the anti-aging drug, a good treatment effect is achieved, i.e. combination of ICIs with the anti-aging drug is superior to the current treatment scheme of ICIs treatment, ICIs in combination with chemotherapy, etc.

In another aspect, a use of immune checkpoint inhibitors in combination with anti-aging drugs in preparation tumor treatment products is further provided according to the present invention. By selecting ICIs in combination with the anti-aging drug, the anti-tumor response is improved and the survival rate of patients can be better improved. Improving the aging of an immune system thus is a new strategy proposed according to the present invention to improve the effect of tumor treatment.

In still another aspect, a use of anti-aging drugs in preparation of drugs for enhancing sensitivity to immune checkpoint inhibitor treatment for tumors.

According to at least one embodiment, the tumor includes head and neck squamous cell carcinoma, bladder cancer and/or breast cancer.

According to at least one embodiment, the immune checkpoint inhibitor includes a PD-1 antibody, a PD-L1 antibody and/or a CTLA-4 antibody.

According to at least one embodiment, the anti-aging drug includes Dasatinib and/or Quercetin.

In still another aspect, a drug composition for treating tumors is further provided according to the present invention, which includes anti-aging drugs and immune checkpoint inhibitors.

According to at least one embodiment, the anti-aging drug includes Dasatinib and/or Quercetin. That is, the anti-aging drug is any one selected from a group consisting of Dasatinib, Quercetin and a combination thereof.

According to at least one embodiment, an immune checkpoint includes PD-1, PD-L1 and/or CTLA-4. That is, the immune checkpoint is any one selected from a group consisting of PD-1, PD-L1, CTLA-4 and combinations thereof.

According to at least one embodiment, the immune checkpoint inhibitor is an antibody specifical binding to the immune checkpoint.

According to at least one embodiment, the immune checkpoint inhibitor includes a PD-1 antibody, a PD-L1 antibody and/or a CTLA-4 antibody. That is, the immune checkpoint inhibitor is any one selected from a group consisting of the PD-1 antibody, the PD-L1 antibody, the CTLA-4 antibody and combinations thereof.

According to at least one embodiment, the tumors include head and neck squamous cell carcinoma, bladder cancer, breast cancer, melanoma, lung cancer, colorectal cancer, prostate cancer, thyroid cancer, brain cancer, esophageal cancer, skin cancer, thymus cancer, stomach cancer, colon cancer, liver cancer, ovarian cancer, uterus cancer, rectal cancer, gallbladder carcinoma, biliary tract cancer and/or pancreatic cancer. That is, the tumor is any one selected from a group consisting of head and neck squamous cell carcinoma, bladder cancer, breast cancer, melanoma, lung cancer, colorectal cancer, prostate cancer, thyroid cancer, brain cancer, esophageal cancer, skin cancer, thymus cancer, stomach cancer, colon cancer, liver cancer, ovarian cancer, uterus cancer, rectal cancer, gallbladder carcinoma, biliary tract cancer, pancreatic cancer and combinations thereof.

According to at least one embodiment, the tumors include head and neck squamous cell carcinoma, bladder cancer and/or breast cancer. The head and neck squamous cell carcinoma specifically include oral cancer, pharyngeal cancer, laryngeal cancer, etc.

In still another aspect, an anti-tumor drug is further provided according to the present invention, which includes the above-mentioned drug composition and a pharmaceutically acceptable excipient.

In still another aspect, a kit for tumor treatment is further provided according to the present invention, which includes a first container, in which anti-aging drugs or a drug containing an anti-aging active ingredient are received, a second container, in which anti-immune checkpoint inhibitors or a drug containing the anti-immune checkpoint inhibitor are received.

According to at least one embodiment, the immune checkpoint includes PD-1, PD-L1 and/or CTLA-4.

According to at least one embodiment, the immune checkpoint inhibitor comprises a PD-1 antibody, a PD-L1 antibody and/or a CTLA-4 antibody.

Compared with the existing treatment scheme, the present invention has the following beneficial effects. The treatment according to the present invention is to treat various solid tumors applying ICIs in combination with the anti-aging drug. Treatment effect obtained is superior to that of current treatment of ICIs treatment alone, ICIs in combination with chemotherapy, etc. In the action process of ICIs in combination with the anti-aging drug, aging cells can be selectively removed, the aging state of an immune system thus is reversed, thereby reducing the inhibitory effect of tumor on the immune micro-environment, and improving the treatment effect on various solid tumors. Further, under the effect of the immune checkpoint inhibitor itself, the treatment effect on the solid tumor is remarkably improved. In addition, the anti-aging drug applied in the present invention has lower toxic and side effects than chemotherapeutic drugs, which can reduce the occurrence rate of adverse reactions, guarantees a better physiological function and quality of life for patients, and enables patients to benefit from ICIs treatment to a greater extent. Moreover, there is no report on this treatment scheme at present, the technical solution disclosed in the present invention thus can provide new strategies and new ideas for further improving the clinical efficacy and prognosis of solid tumors.

### BRIEF DESCRIPTION OF THE DRAWINGS

FIG. 1 shows two independent experiments, 4NQO chemically-induced head and neck squamous cell carcinoma model C57 wild-type mice being divided into four groups for drug administration in each experiment, A showing the survival time of mice with head and neck squamous cell carcinoma treated with Isotype+Vehicle (control group), Anti PD-L1, Anti PD-L1 in combination with cisplatin, and Anti PD-L1 in combination with D+Q, respectively; B showing the percent survival of mice with head and neck squamous cell carcinoma treated with Isotype+Vehicle (control group), Anti PD-1, Anti PD-1 in combination with cisplatin, and Anti PD-1 in combination with D+Q, respectively.

FIG. 2 shows a tumor picture of mice obtained after dividing bladder cancer xenograft models constructed by injecting MB49 cells into the armpits of C57 mice into four groups and respectively administering Isotype+Vehicle (control group), Anti PD-L1, Anti PD-L1 in combination with cisplatinp, and Anti PD-L1 in combination with D+Q for treatment.

FIG. 3 shows three independent experiments, three MB49 bladder cancer xenograft models being respectively constructed, and dividing mice into four groups for drug administration for each experiment, A showing a tumor growth curve of mice after being treated with Isotype+Vehicle (control group), Anti PD-L1, Anti PD-L1 in combination with cisplatin, and Anti PD-L1 in combination with D+Q; B showing a tumor growth curve of mice after being treated with Isotype+Vehicle (control group), Anti PD-1, Anti PD-1 in combination with cisplatin, and Anti PD-1 in combination with D+Q; C. a tumor growth curve of mice after being treated with Isotype+Vehicle (control group), Anti CTLA-4, Anti CTLA-4 and cisplatin, and Anti CTLA-4 in combination with D+Q.

FIG. 4 shows three independent experiments, three E0771 breast cancer xenograft models being respectively constructed, and dividing mice into four groups for drug administration for each experiment, A showing a tumor growth curve of mice after being treated with Isotype+Vehicle (control group), Anti PD-L1, Anti PD-L1 in combination with cisplatin, and Anti PD-L1 in combination with D+Q, and B showing a tumor growth curve of mice after being treated with Isotype+Vehicle (control group), Anti PD-1, Anti PD-1 in combination with cisplatin, and Anti PD-1 in combination with D+Q; C. a tumor growth curve of mice after being treated with Isotype+Vehicle (control group), Anti CTLA-4, Anti CTLA-4 in combination with cisplatin, and Anti CTLA-4 in combination with D+Q.

FIG. 5 shows the percent survival of the four groups of mice in the bladder cancer xenograft model experiment in FIG. 3).

FIG. 6 shows: the percent survival of the four groups of mice in the breast cancer xenograft model experiment in FIG. 4.

### DETAILED DESCRIPTION

It should be noted that the following detailed description is exemplary and intended to provide further explanation of the present invention. Unless otherwise defined, all technical and scientific terms used herein have the same meaning as commonly understood by one of ordinary skill in the art to which the present invention belongs.

It should be noted that the term used herein is for the purpose of describing specific embodiments only, and is not intended to limit the exemplary embodiments according to the present invention. As used herein, unless the context clearly dictates otherwise, the singular form is intended to include the plural form as well, furthermore, it should also be understood that when the terms “contain” and/or “include” are used in this description, it indicates that there are features, steps, operations, devices, assemblies and/or combinations thereof.

The present invention will now be further described with reference to specific examples. The following embodiments are only for explaining the present invention, but do not constitute a limitation to the present invention. The test samples and test procedures used in the following embodiments include the following content (if the specific experimental conditions are not specified in the embodiments, it usually follows the conventional conditions, or follows the conditions recommended by the reagent company; reagents, consumables, etc., used in the following embodiments can be obtained from commercial sources unless otherwise specified).

I. Effects of ICIs (Immune Checkpoint Inhibitors) in Combination with Anti-Aging Drugs on Chemically-Induced Mouse Head and Neck Squamous Cell Carcinoma

A 4-NQO chemically-induced mouse model was used in this experiment. Forty 6-8 week-old wild-type C57 mice (Gem Pharma, Jiangsu) were purchased and fed in SPF barriers system, and 4-nitroquinoline-N-oxide (hereinafter referred to as 4-NQO) (Sigma Aldrich, N8141, 5G) was dissolved in polyethylene glycol at 500 mg/kg for use. When 4-NQO was used, it was added to the drinking water of the mice to make the concentration thereof at 50 mg/kg. 4-NQO at 50 mg/kg is continuously fed to the mice for 16 weeks, then ordinary purified water is fed to the mice for about 18 weeks. The tongue and oral mucosa of the mice were taken for HE section to confirm the occurrence of head and neck squamous cell carcinoma.

In Experiment 1, 20 mice with head and neck squamous cell carcinoma were constructed and divided into 4 groups, with 5 mice in each group. The four groups of mice were treated with drugs of (1) Isotype (derived from BioXcell, BE0090, homotype antibody control)+Vehicle (solvent), (2) Anti PD-L1 (BioXcell, BE0361), (3) Anti PD-L1 in combination with cisplatin (Anti PD-L1 being derived from BioXcell, BE0361; cisplatin being derived from Sigma Aldrich, PHR1624), and (4) Anti PD-L1 in combination with Dasatinib and Quercetin (Anti PD-L1 being derived from BioXcell, BE0361; Dasatinib being derived from Selleck, S1021; Quercetin being derived from Selleck, S2391; in the following contents, Dasatinib+Quercetin will be referred to as D+Q). Anti PD-L1 is injected intraperitoneally once a week, with a single injection dose of 200 ug/mouse. Cisplatin was dissolved in PBS, and the dose of cisplatin for single intraperitoneal injection was 2 mg/kg, twice a week. The single dose of D+Q was 5/50 mg/kg, i.e. a single dose of Dasatinib and Quercetin is respectively 5 mg/kg 50 mg/kg, by oral gavage, twice a week.

In Experiment 2, 20 mice with head and neck squamous cell carcinoma were constructed and divided into 4 groups, with 5 mice in each group, and the four groups of mice were treated with drugs respectively. Unlike the first experiment, Anti PD-L1 was replaced with Anti PD-1 in each group (Anti PD-1 being derived from BioXcell, BE0273). Namely, the 4 groups were treated with drugs of Isotype+Vehicle, Anti PD-1, Anti PD-1 in combination with cisplatin, and Anti PD-1 in combination with D+Q respectively. Similarly, Anti PD-1 was injected intraperitoneally once a week with a single injection dose of 200 ug/mouse. The remaining drug sources and treatment methods were the same as those in Experiment 1.

In Experiment 1 and Experiment 2, the percent survival of the mice with head and neck squamous cell carcinoma after drug treatment was recorded, which was used as the evaluation standard of the treatment effect. Specifically, mouse death was considered as the humane endpoint of the mice (that is, when the subcutaneous tumor reached 1500 mm³ in size, it is considered that the mice were dead).

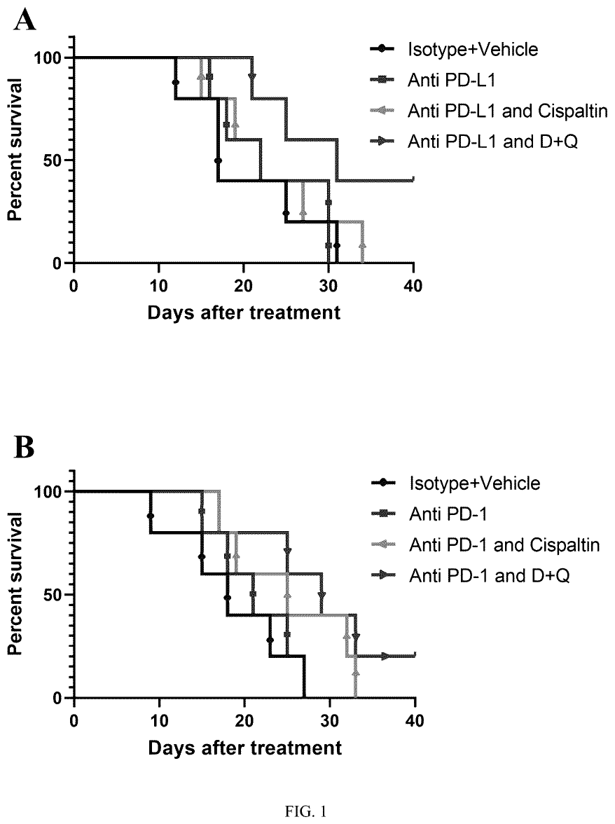

The results are as shown in FIG. 1. In all experimental groups administered in Experiment 1 and Experiment 2, compared with administration of the immune checkpoint inhibitor alone or the immune checkpoint inhibitor in combination with cisplatin, the mice with head and neck squamous cell carcinoma treated with the immune checkpoint inhibitor (Anti PD-L1 or Anti PD-1) in combination with the anti-aging drug (D+Q) had a greater percent survival, indicating that Anti PD-L1 in combination with the anti-aging drug or Anti PD-1 in combination with the anti-aging drug D+Q has a better anti-tumor effect than administration of ICIs alone or ICIs in combination with cisplatin. That is, the treatment method of the immune checkpoint inhibitor in combination with the anti-aging drug has a significantly better anti-tumor effect than the treatment method of administration of the immune checkpoint inhibitor alone or the immune checkpoint inhibitor in combination with cisplatin.

II. Effects of ICIs in Combination with Anti-Aging Drugs on Mouse Bladder Cancer and Breast Cancer Xenografts

(I) Mouse Bladder Cancer Model Experiment

1. Experiment I: 20 C57 mice were divided into 4 groups, with 5 mice in each group, 2×10⁶ MB49 cells (Meisen Cell, Zhejiang) (cell culture conditions: DMEM high glucose medium, 10% FBS, and 1% antibiotics)) were injected into the armpits of 20 C57 mice, and the tumor growth of the mice was observed every day. When the tumor of the mice was about 100 mm³ in size, the four groups of mice with bladder cancer were treated with Isotype+Vehicle (control group), Anti PD-L1, Anti PD-L1 in combination with cisplatin, and Anti PD-L1 in combination with D+Q respectively, the administration dosage and method are the same as those in the aforementioned 4NQO head and neck squamous cell carcinoma model experiment, the tumor size was measured with a vernier caliper every three days, and the tumor volume was calculated (the formula is: ½ length×width²).

2. Experiment II: The same number of C57 mice were used and divided into 4 groups in the same way as in Experiment 1 to construct a bladder cancer model. Specifically, during the administration process, Anti PD-L1 in each group in Experiment 1 was replaced with Anti PD-1, and the rest were the same as in Experiment 1. That is, in Experiment 2, the four groups of mice with bladder cancer were treated with Isotype+Vehicle (control group), Anti PD-1, Anti PD-1 in combination with cisplatin, and Anti PD-1 in combination with D+Q respectively.

3. Experiment III: The same number of C57 mice were used and divided into 4 groups in the same way as in Experiment 1 to construct a bladder cancer model. Specifically, during the administration process, Anti PD-L1 in each group in Experiment 1 was replaced with Anti CTLA-4, and the rest were the same as in Experiment 1. That is, in Experiment 3, the four groups of mice with bladder cancer were treated with Isotype+Vehicle (control group), Anti CTLA-4, Anti CTLA-4 in combination with cisplatin, and Anti CTLA-4 in combination with D+Q respectively.

(II) Mouse Breast Cancer Model Experiment

Experiment I: 20 C57 mice were divided into 4 groups, with 5 mice in each group, 5×10⁶ E0771 cells (Meisen Cell, Zhejiang) (cell culture conditions: DMEM high glucose medium, 10% FBS and 1% antibiotics) were injected into the armpits of 20 C57 mice, and the tumor growth of the mice was observed every day. When the tumor of the mice was about 100 mm³ in size, the four groups of mice with breast cancer were treated with Isotype+Vehicle (control group), Anti PD-L1, Anti PD-L1 in combination with cisplatin, and Anti PD-L1 in combination with D+Q respectively, the administration dosage and method are the same as those in the aforementioned 4NQO head and neck squamous cell carcinoma model experiment, the tumor size was measured with a vernier caliper every three days, and the tumor volume was calculated (the formula is ½ length×width²).

2. Experiment II: The same number of C57 mice were used and divided into 4 groups in the same way as in Experiment 1 to construct a breast cancer model. Specifically, during the administration process, Anti PD-L1 in each group in Experiment 1 was replaced with Anti PD-1, and the rest were the same as in Experiment 1. That is, in Experiment 2, the four groups of mice with breast cancer were treated with Isotype+Vehicle (control group), Anti PD-1, Anti PD-1 in combination with cisplatin, and Anti PD-1 in combination with D+Q respectively.

3. Experiment III: The same number of C57 mice were used and divided into 4 groups in the same way as in Experiment 1 to construct a breast cancer model. Specifically, during the administration process, Anti PD-L1 in each group in Experiment 1 was replaced with Anti CTLA-4, and the rest were the same as in Experiment 1. That is, in Experiment 3, the four groups of mice with breast cancer were treated with Isotype+Vehicle (control group), Anti CTLA-4, Anti CTLA-4 in combination with cisplatin, and Anti CTLA-4 in combination with D+Q respectively.

Tumor size data measured in the above experiments were drawn into a tumor growth curve graph. Statistical analysis was performed using GarphPad Prism 8.3.0 analysis, and using two-way analysis of variance (Two-way ANOVA), Error-bars: standard error of the mean.

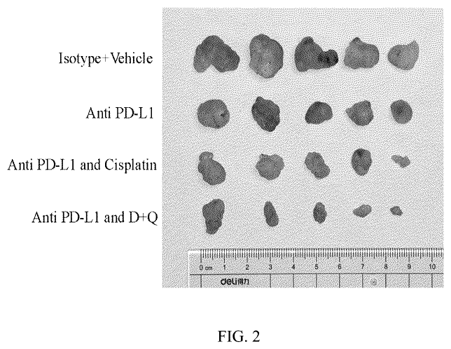

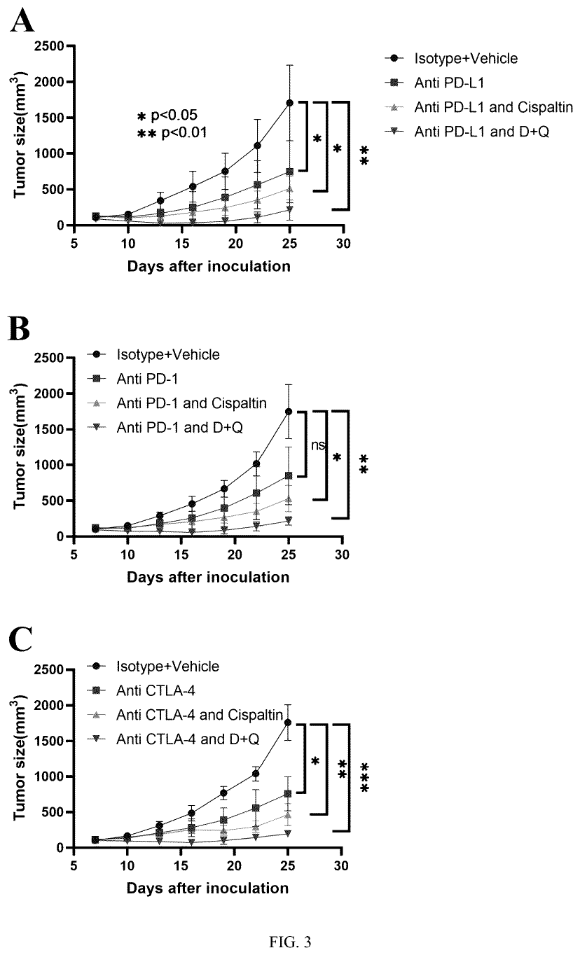

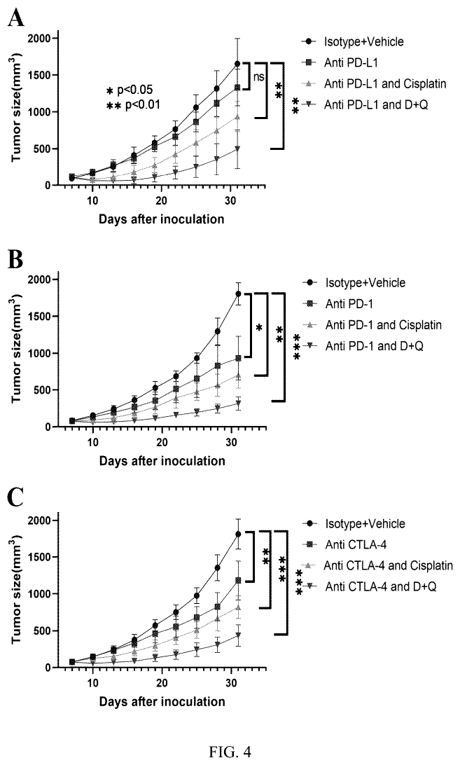

It is found that the mice with cancer treated with the immune checkpoint inhibitors (Anti PD-L1/Anti PD-1/Anti CTLA-4) in combination with the anti-aging drug (D+Q) have the smallest tumors among the four groups of treatment methods, which is consistent on the bladder cancer and breast cancer mouse models in this experiment, confirming that the immune checkpoint inhibitors (ICIs) in combination with the anti-aging drug D+Q have a better anti-tumor effect than treatment using ICIs alone or treatment using ICIs in combination with cisplatin. As shown in FIG. 2 and FIG. 3, in the bladder cancer xenograft model, the treatment effect of ICIs in combination with the anti-aging drug D+Q is significant, and is significantly better than that in other groups. As shown in FIG. 4, in the breast cancer xenograft model, the treatment effect of ICIs in combination with the anti-aging drug D+Q is significant, and is significantly better than that in other groups.

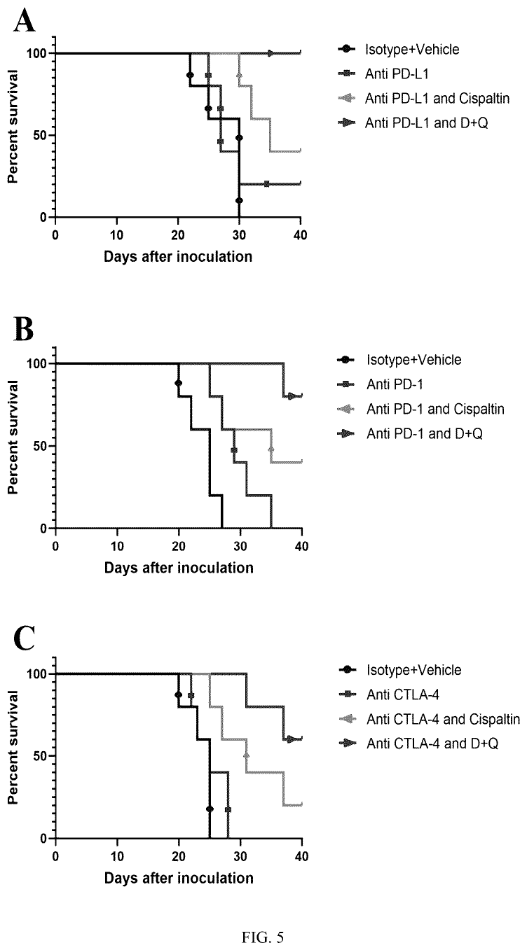

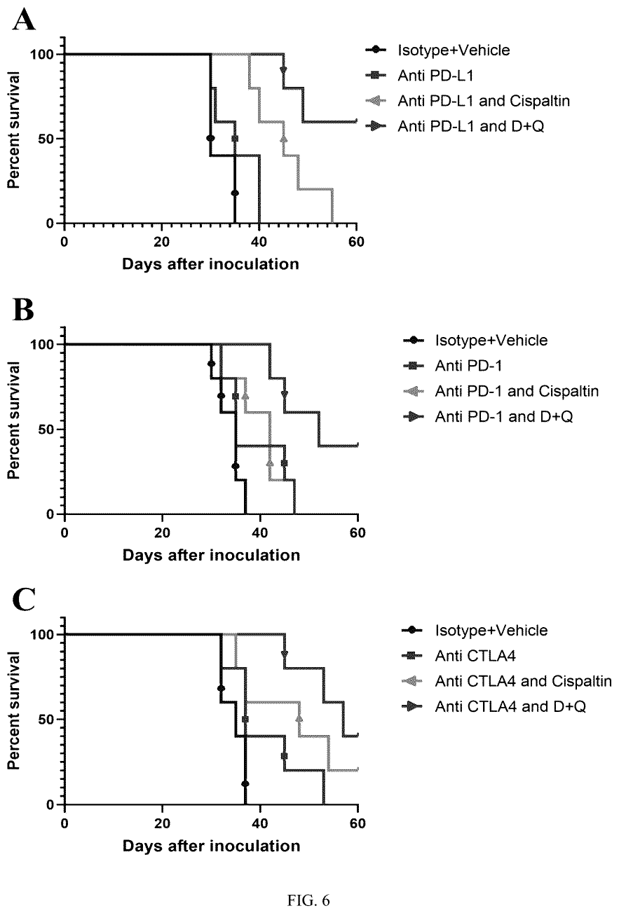

Meanwhile, this experiment also recorded the survival condition of the mice with bladder cancer and breast cancer after treatment. Specifically, mouse death was considered as the humane endpoint of the mice (that is, when the subcutaneous tumor reached 1500 mm³ in size, it is considered that the mice were dead). The survival curve graph of the mice was drawn as shown in FIG. 5 and FIG. 6. It was found that the survival rate of the mice with cancer treated with ICIs (Anti PD-L1/Anti PD-1/Anti CTLA-4) in combination with the anti-aging drug D+Q was significantly greater than that in the remaining three groups, and this point was consistent on the mice with bladder cancer and breast cancer in this experiment, proving that ICIs in combination with the anti-aging drug D+Q have a better anti-tumor effect than administration of ICIs alone or ICIs in combination with cisplatin, and the survival time of individuals suffering from cancer can be prolonged.

Obviously, the above-mentioned embodiments of the present invention are only examples for clearly illustrating the technical solutions of the present invention, and are not intended to limit the specific embodiments of the present invention. Any modifications, equivalent replacements, improvements, etc. made within the spirit and principles of the claims of the present invention shall be included within the protection scope of the claims of the present invention.

1. Use of immune checkpoint inhibitors in combination with anti-aging drugs in preparation of tumor treatment products.

2. Use of anti-aging drugs in preparation of drugs for enhancing sensitivity to tumor immune checkpoint inhibitor treatment.

3. A drug composition for treatment of tumors, comprising anti-aging drugs and immune checkpoint inhibitors.

4. The drug composition according to claim 3, wherein the anti-aging drug comprises Dasatinib and/or Quercetin.

5. The drug composition according to claim 3, wherein an immune checkpoint comprises PD-1, PD-L1 and/or CTLA-4.

6. The drug composition according to claim 3, wherein the immune checkpoint inhibitor is an antibody specific binding to the immune checkpoint.

7. The drug composition according to claim 3, wherein tumors comprise head and neck squamous cell carcinoma, bladder cancer, breast cancer, melanoma, lung cancer, colorectal cancer, prostate cancer, thyroid cancer, brain cancer, esophageal cancer, skin cancer, thymus cancer, stomach cancer, colon cancer, liver cancer, ovarian cancer, uterus cancer, rectal cancer, gallbladder carcinoma, biliary tract cancer and/or pancreatic cancer.
